(19) United States
(12) Patent Application Publication
(10) Pub. No.: US 2025/0375471 A1
(43) Pub. Date: Dec. 11, 2025

(54) HEPARANASE INHIBITORS AND METHODS OF USE

(71) Applicant: The Board of Regents of the University of Oklahoma, Norman, OK (US)

(72) Inventors: Paul L. DEANGELIS, Edmond, OK (US); Dixy E. GREEN, Edmond, OK (US)

(21) Appl. No.: 18/876,815

(22) PCT Filed: Jun. 23, 2023
(86) PCT No.: PCT/US2023/026124

§ 371 Date: Dec. 19, 2024

Related U.S. Application Data
(60) Provisional application No. 63/355,144, filed on Jun. 24, 2022.

(51) Int. Cl. A61K 31/726; A61P 7/02; C08B 37/00; C08L 5/08

(52) U.S. Cl. CPC A61K 31/726; A61P 7/02; C08B 37/0063; C08L 5/08

(57) ABSTRACT

Polymers comprising the repeat structure [4-D-glucuronic acid-α1,4-D-N-acetylglucosamine-α1-]ₙ ([-4-D-GlcUA-α1,4-D-GlcNAc-α1-]ₙ) are disclosed, wherein at least one sulfur moiety is linked to at least one hydroxyl of the repeat structure. Also disclosed are methods of producing and using these polymers.

### CROSS-REFERENCE TO RELATED APPLICATIONS

The present application claims priority under 35 U.S. C. § 119(e) to U.S. provisional Application No. 63/355,144, filed Jun. 24, 2022. The entire contents of the above-referenced patent application(s) are hereby expressly incorporated herein by reference.

### BACKGROUND

Glycosaminoglycans (GAGs) are linear, negatively charged heteropolysaccharides that are essential components of the extracellular matrix (ECM) and contribute to their biological and biomechanical properties (1). One of the most studied classes of GAGs is heparin/heparan sulfate (HS) with backbones containing disaccharide repeats comprised of (i) a hexosamine residue, glucosamine (GlcN) with an N-acetyl (Ac) or N-sulfo group, and (ii) a uronic acid residue, either glucuronic acid (GlcUA) or iduronic acid (2). The specific biological roles of GAGs, such as modulating cell-cell interactions, enzyme activity, and cell proliferation during various processes are related to their backbone structure, post-polymerization modifications (e.g., position-specific sulfation, epimerization), chain size, and their cellular localization (3). Several methods are available to synthesize various natural and artificial GAG structures with bioactivities have been employed in the past (4-6, 15).

HS is broken down by heparanase, an endo-beta-glucuronidase. Overexpression of heparanase is significantly correlated with cancer metastases. Heparanase is thus a therapeutic target in the treatment of metastatic cancer. Heparosan (HEP; [→4)-α-D-GlcUA-(1→4)-β-D-GlcNAc(1→]ₙ) is the unsulfated biosynthetic precursor to HS and heparin in animals as well as the capsular polysaccharide of certain pathogenic microbes (FIG. 1). New drugs with improved potency and/or selectivity against heparanase are desirable.

Heparin and some derivatives thereof act as heparanase inhibitors; therefore, an initial approach in the field was to use modified polymers that resemble the HS substrate of heparanase. The drug heparin (a highly sulfated version of HS) is a strong heparanase inhibitor, but due to its potent anticoagulant activity, it must be ‘de-activated’ by chemical treatments (i.e., periodate, desulfation) to avoid hemorrhagic side effects if used for cancer treatment. Some heparin-like derivatives have been tested in cancer treatment clinical trials, including Muparfostat (a mix of sulfated di- to hexasaccharides), PG545 (a highly sulfated hexasaccharide with a lipophilic group), Roneparstat (a fully N-acetylated glycol-split heparin; see, for example, U.S. Pat. No. 7,781,416), and Necuparanib (a glycol-split low molecular weight heparin). However, these heparin-like derivatives are complex heterogeneous mixtures (i.e., 1-3 sulfates/repeat unit, variable acetylation and epimerization, etc.) derived from natural sources; therefore, the QC methods for characterization are complex, and it is costly to monitor batch-to-batch or seasonal variations. In addition, animal sourced materials (e.g., porcine intestinal mucosa) have supply chain security issues; for example, lots of intentionally contaminated heparin in 2008 led to deaths and fatalities.

Furthermore, both PG545 and Necuparanib also retain significant anticoagulant activity, and Roneparstat requires high dosage levels because of its short half-life; all of which are liabilities that can limit their clinical usage. PG545 (currently one of the most potent of the described inhibitors), lacks significant anticoagulant activity and cell toxicity, but it has other undesired effects, including immune cell effects that are cell type dependent (some inhibitory, some stimulatory).

The vast majority of the current heparin mimics have the disadvantage that they are not specific for heparanase and likely interact with different heparin-binding proteins, with unknown consequences and off target effects. In addition, three of the four mimics in clinical trials were heterogeneous in their structure, adding further to their uncertainty as viable drugs for use in humans. A number of heparanase-inhibiting small molecules were reported, but none entered clinical testing.

Therefore, there is a need in the art for new and improved compositions made with defined and safe precursors that possess heparanase inhibitory activity while avoiding the side effects of the current heparin-like derivatives.

### BRIEF DESCRIPTION OF THE DRAWINGS

FIG. 1 shows structures of heparosan and testosteronan. The only difference between the two unsulfated sugar polymers is the conformation of one of two the glycosidic linkages that form the co-polymer repeat (marked with arrow).

FIG. 2 shows an agarose gel (1.5%, 1×TAE) comparison of Testan and sulfated Testan. Chemical sulfation of Testan (T; here the product of bacterial fermentation) adds negative charges to the polymer backbone; therefore, sulfated Testan (sT; here the result of chemical sulfation) migrates faster than non-sulfated Testan. Additionally, the band color with the Stains All detection reagent shifts from blue to purple upon sulfation. The synthesis is reproducible as the two independent batches of the sTestan prototype shown here (1 or 2) appear equivalent.

FIG. 3 shows a human heparanase enzyme (Hepase) challenge of chemically sulfated testosteronan (sTestan). Polyacrylamide gel electrophoresis (PAGE) analysis (20% gel, 1×TBE with Stains-All detection) demonstrates that sTestan (180 ng) was recalcitrant to the action of heparanase (73 ng) even after 24 hours of digestion. In contrast, the sulfated synthetic heparosan or natural heparan sulfate polymers were cleaved in minutes with much less of the enzyme (as in FIGS. 6 and 8).

FIG. 4 shows a gel analysis (1.5% agarose, 1×TAE, Stains-All detection) of Testan (T) and various sulfated derivatives (sTestan; sT). The asterisk denotes samples tested in various bioassays. The color change from blue to purple to yellow/orange indicates zero/low, medium, or high sulfation levels, respectively.

FIG. 5 shows an NMR of chemical aqueous Testan. The 1D NMR shows that H2 of GlcA of Testan (red) is strongly shifted upfield after modification to aqueous sTestan (blue), indicating that the C2-OH position is sulfated.

FIG. 6 shows the inhibition of human heparanase by aq sTestan (bacterial fermented Testan sulfated by the water-based method) or by anhydrous sTestan (anh sT; bacterial fermented Testan sulfated with the dry solvent method). PAGE (15%, 1×TBE) analysis of the kinetics of Hepase cleavage of a fluorescent sulfo-O-linked heparosan substrate (starting material marked with arrow) with a titration of the sTestan inhibitor (decreasing amounts in nanograms from left to right; 15-min time point shown here). A calibrant series of fluorescent substrate treated with varying amounts of heparanase enzyme (‘¼,’ ‘½,’ or ‘1’ for 25%, 50%, or 100% relative amounts, respectively) was used to evaluate inhibitor potency (shown on left). Both sTestan formulations are competitive inhibitors of human heparanase.

FIG. 7 shows the inhibition of human heparanase by aq sTestan (synthetic chemoenzymatically prepared Testan sulfated by the aqueous method). PAGE (15%) analysis of the kinetics of Hepase cleavage of a fluorescent sulfo-O-linked heparosan substrate (starting material marked with arrow) with a titration of the sTestan inhibitor (increasing amounts in nanograms from left to right; 15-min time point shown here) or a known heparanase inhibitor (generic Roneparstat). The sTestan is a competitive inhibitor of human heparanase.

FIG. 8 shows the inhibition of human heparanase digestion of a natural heparan sulfate (HS) substrate by aq sTestan. The sTestan polymer could inhibit the degradation of the HS substrate by human heparanase at various substrate to inhibitor ratios (Sub/Inhib) at the 30 min time-point.

FIG. 9 shows results of an anticoagulant assay using heparin, aq sTestan, or generic Roneparstat (Rone). A chromogenic assay (based on the thrombin/antithrombin III interaction and bioactivity) that is used diagnostically to assess heparin levels in human blood was used to compare the international heparin standard (black diamonds) to sTestan (black triangle) and Roneparstat (gray circles). The arrow marks the value of the uninhibited control with maximum coagulation in this assay. The sTestan has no anticoagulant activity even at 20,000-fold higher concentrations than heparin. Furthermore, sTestan has less anticoagulant activity than Roneparstat. These findings indicate that sTestan is unlikely to cause bleeding side effects.

FIG. 10 shows the relative effect of two types of sTestan (aqueous or anhydrous reactions) versus heparin or Roneparstat in an assay for heparin-induced thrombocytopenia (HIT). The heparin standard (black diamonds) in solution competed well with plate-bound heparin for making the HIT antibody complex, thus reducing the assay signal derived from bound antibody. The aqueous sTestan (black triangles) and Roneparstat (gray circles) were less reactive, as it takes more competitor molecules to reduce the signal. The aq sTestan did not form the HIT complex with Platelet Factor 4 and antibody as readily as the drug heparin or Ronepartsat, and is therefore less likely to cause side effects.

FIG. 11 shows the ability of aq sTestan to block cancer cell metastasis in a trans-well migration assay. The level of inhibition of cell invasion was comparable to or slightly better than Roneparstat, a current best-in-field drug candidate targeting heparanase.

FIG. 12 shows production of a sTestan-heparosan chimeric polysaccharide using PAGE analysis (6% gel with Stains-All detection). A new band with purple staining at a higher molecular weight (denoted with an arrow) indicates the addition of a heparosan tail to the sTestan chain using heparosan synthase and UDP-sugars. This non-optimized example shows that a subset of sTestan was modified, not the entire population.

### DETAILED DESCRIPTION

Described herein is a novel polysaccharide class, sulfated or sulfonated testosteronan (also referred to herein as sTestoteronan or sTestan). Testosteronan (also referred to herein Testan, and first disclosed in International Patent Application Publication No. WO 2012/16353) is a polysaccharide having the repeat structure [-4-D-glucuronic acid-α1,4-D-N-acetylglucosamine-α1-]ₙ ([-4-D-GlcUA-α1,4-D-GlcNAc-α1-]ₙ). The sulfated or sulfonated sTestan of the present disclosure is recalcitrant to digestion by heparanase, an enzyme important in human health and disease. Furthermore, sTestan acts as a competitive inhibitor of heparanase and thus can serve as a treatment for cancer or other diseases with heparanase involvement, such as (but not limited to) diabetes, a complication of diabetes (e.g., cardiomyopathy), atherosclerosis, thrombosis, a viral infection (e.g., herpes simplex), and the like. In addition, sTestan can be used as a selective modulator of sugar protein interactions due to its unique structural difference with naturally occurring polymers in the body.

Before further describing various embodiments of the compositions and methods of the present disclosure in more detail by way of exemplary description, examples, and results, it is to be understood that the embodiments of the present disclosure are not limited in application to the details of methods and compositions as set forth in the following description. The embodiments of the compositions and methods of the present disclosure are capable of being practiced or carried out in various ways not explicitly described herein. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the embodiments of the present disclosure may be practiced without these specific details. In other instances, features which are well known to persons of ordinary skill in the art have not been described in detail to avoid unnecessary complication of the description. While the compositions and methods of the present disclosure have been described in terms of particular embodiments, it will be apparent to those of skill in the art that variations may be applied to the compositions and/or methods and in the steps or in the sequence of steps of the method described herein without departing from the concept, spirit, and scope of the inventive concepts as described herein. All such similar substitutes and modifications apparent to those having ordinary skill in the art are deemed to be within the spirit and scope of the inventive concepts as disclosed herein.

All patents, published patent applications, and non-patent publications referenced or mentioned in any portion of the present specification, including but not limited to U.S. Pat. Nos. 9,695,427, and 10,273,517, are indicative of the level of skill of those skilled in the art to which the present disclosure pertains, and are hereby expressly incorporated by reference in their entirety to the same extent as if the contents of each individual patent or publication was specifically and individually incorporated herein.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” and “approximately” are used to indicate that a value includes the inherent variation of error for the composition, the method used to administer the composition, or the variation that exists among the objects, or study subjects. As used herein the qualifiers “about” or “approximately” are intended to include not only the exact value, amount, degree, orientation, or other qualified characteristic or value, but are intended to include some slight variations due to measuring error, observer error, wear and tear, and combinations thereof, for example. The term “about” or “approximately”, where used herein when referring to a measurable value such as an amount, percentage, temporal duration, and the like, is meant to encompass, for example, variations of ±20%, or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment. Further, all references to one or more embodiments or examples are to be construed as non-limiting to the claims.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50, as well as 1.1, 1.2, 1.3, 1.4, 1.5, etc., 2.1, 2.2, 2.3, 2.4, 2.5, etc., and so forth, where the range is not limited solely to integers. Reference to a series of ranges includes ranges which combine the values of the boundaries of different ranges within the series. Thus, to illustrate reference to a series of ranges, for example, a range of 1-1,000 includes, for example, 1-10, 10-20, 20-30, 30-40, 40-50, 50-60, 60-75, 75-100, 100-150, 150-200, 200-250, 250-300, 300-400, 400-500, 500-750, 750-1,000, and includes ranges of 1-20, 10-50, 50-100, 100-500, and 500-1,000. The range 100 units to 2000 units therefore refers to and includes all values or ranges of values of the units, and fractions of the values of the units and integers within said range, including for example, but not limited to 100 units to 1000 units, 100 units to 500 units, 200 units to 1000 units, 300 units to 1500 units, 400 units to 2000 units, 500 units to 2000 units, 500 units to 1000 units, 250 units to 1750 units, 250 units to 1200 units, 750 units to 2000 units, 150 units to 1500 units, 100 units to 1250 units, and 800 units to 1200 units. Any two values within the range of about 100 units to about 2000 units therefore can be used to set the lower and upper boundaries of a range in accordance with the embodiments of the present disclosure.

The term “GlcNAc” as used herein refers to N-acetylglucosamine. The terms “GlcA” and “GlcUA” as used herein are interchangeable and refer to glucuronic acid. The terms “UDP-GlcNAc” and “UDP-GlcUA” refer to uridine diphosphate sugar precursors of GlcNAc and GlcUA, respectively. These compounds are used by glycosyltransferases to transfer GlcNAc/GlcUA residues to substrates. In the formula “[-4-D-GlcUA-α1,4-D-GlcNAc-α1-]ₙ,” n may be in a range of, for example, 1-100, or 2-100, or 1-200, or 2-200, or 1-500, 2-500, or 1-1000, or any range inclusive therein.

The term “pharmaceutically acceptable” refers to compounds and compositions which are suitable for administration to humans and/or animals without undue adverse side effects such as toxicity, irritation and/or allergic response commensurate with a reasonable benefit/risk ratio.

The term “active agent” as used herein is intended to refer to a substance which possesses a biological activity relevant to the present disclosure, and particularly refers to therapeutic and diagnostic substances which may be used in methods described in the present disclosure. By “biological activity” is meant the ability to act on or modify an organic or inorganic molecule, or the molecular, biochemical, or physiological system of a cell, tissue, or organism without reference to how the active agent has its effects. “Bioactivity” refers to any biological property of an active agent.

As used herein, “pure,” “substantially pure,” or “isolated” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species (e.g., the peptide compound) is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure. Where used herein the term “high specificity” refers to a specificity of at least 90%, or at least 91%, or at least 92%, or at least 93%, or at least 94%, or at least 95%, or at least 96%, or at least 97%, or at least 98%, or at least 99%. Where used herein the term “high sensitivity” refers to a sensitivity of at least 90%, or at least 91%, or at least 92%, or at least 93%, or at least 94%, or at least 95%, or at least 96%, or at least 97%, or at least 98%, or at least 99%.

The terms “subject” and “patient” are used interchangeably herein and will be understood to refer an organism to which the compositions of the present disclosure are applied and used, such as a vertebrate or more particularly to a warm-blooded animal, such as a mammal. Non-limiting examples of animals within the scope and meaning of this term include dogs, cats, rats, mice, guinea pigs, chinchillas, horses, goats, cattle, sheep, llamas, zoo animals, Old and New World monkeys, non-human primates, and humans.

“Treatment” refers to therapeutic treatments, such as for healing or restoration of damaged tissues. The term “treating” refers to administering the composition to a patient such therapeutic purposes, and may result in an amelioration of the condition or disease.

The terms “therapeutic composition” and “pharmaceutical composition” refer to an active agent composition, such as the hydrogel compositions described herein, that may be administered to a subject by any method known in the art or otherwise contemplated herein, wherein administration of the composition brings about a therapeutic effect as described elsewhere herein. In addition, certain compositions of the present disclosure may be designed to provide targeted, delayed, controlled, extended, and/or sustained release using formulation techniques which are well known in the art.

The term “effective amount” refers to an amount of an active agent which is sufficient to exhibit a detectable biochemical and/or therapeutic effect, for example without excessive adverse side effects (such as toxicity, irritation, and allergic response) commensurate with a reasonable benefit/risk ratio when used in the manner of the present disclosure. The effective amount for a patient will depend upon the type of patient, the patient's size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by a person of ordinary skill in the art using routine experimentation based on the information provided herein.

The term “ameliorate” means a detectable or measurable improvement in a subject's condition or symptom thereof. A detectable or measurable improvement includes a subjective or objective decrease, reduction, inhibition, suppression, limit or control in the occurrence, frequency, severity, progression, or duration of the condition, or an improvement in a symptom or an underlying cause or a consequence of the condition, or a reversal of the condition. A successful treatment outcome can lead to a “therapeutic effect,” or “benefit” of ameliorating, decreasing, reducing, inhibiting, suppressing, limiting, controlling or preventing the occurrence, frequency, severity, progression, or duration of a condition, or consequences of the condition in a subject.

A decrease or reduction in worsening, such as stabilizing the condition, is also a successful treatment outcome. A therapeutic benefit therefore need not be complete ablation or reversal of the condition, or any one of, most of, or all of the adverse symptoms, complications, consequences or underlying causes associated with the condition. Thus, a satisfactory endpoint may be achieved when there is an incremental improvement such as a partial decrease, reduction, inhibition, suppression, limit, control or prevention in the occurrence, frequency, severity, progression, or duration, or inhibition or reversal of the condition (e.g., stabilizing), over a short or long duration of time (e.g., seconds, minutes, hours).

As used herein, the term “concurrent therapy” is used interchangeably with the terms “combination therapy” and “adjunct therapy,” and will be understood to mean that the patient in need of treatment is treated or given another drug for the disease in conjunction with the novel active agents (e.g., sTestan) of the present disclosure. This concurrent therapy can be sequential therapy where the patient is treated first with one drug and then the other, or the two drugs are given simultaneously. In certain embodiments, the subject may be administered, with the sTestan, an additional therapeutic and/or diagnostic agent. The additional agent may be administered simultaneously, within the same or different compositions, or may be administered sequentially. For example, the sTestan may be administered first and the additional agent administered second. Or the sTestan may be administered after the additional agent is administered.

As used herein, “chemotherapeutic agent” or “chemotherapeutic” can refer to a therapeutic agent utilized to prevent or treat a cancer.

The term “molecular weight,” as used herein, can generally refer to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) with light scattering detection, or electrophoresis with appropriate standards. GPC molecular weights are reported as the weight-average molecular weight (M_{w}) as opposed to the number-average molecular weight (Mₙ).

The term “polysaccharide” as used herein will be understood to refer to large carbohydrate molecules comprising from about 20 sugar units to thousands of sugar units (i.e., monosaccharide residues). The term “oligosaccharide” as used herein will be understood to refer to smaller carbohydrate molecules comprising less than about 20 sugar units. The term “polymer” as used herein will be understood to refer to naturally occurring or synthetic compounds that are made up of repeated units. The term “polymer” encompasses both oligosaccharide and polysaccharide structures.

The term “polydisperse” as used herein refers to a polymer having chain lengths that vary over a wide range of molecular masses such that there is molecular-weight nonhomogeneity. The terms “monodisperse,” “substantially monodisperse,” and “quasi-monodisperse” as used herein will be understood to refer to defined glycosaminoglycan polymers that have a narrow size distribution. In addition, a polydispersity value or heterogeneity index is a measure of the distribution of molecular mass in a given polymer sample. The calculated polydispersity value is the weight average molecular weight divided by the number average molecular weight; it indicates the distribution of individual molecular masses in a batch of polymers. The polydispersity value has a value equal to or greater than 1, but as the polymer chains approach uniform chain length, the polydispersity value approaches unity.

The active agents disclosed herein (e.g., sulfated or sulfonated testosteronans) can be formulated into compositions for delivery to a subject. The composition can be administered alone and/or mixed with a pharmaceutically acceptable vehicle or excipient. Suitable vehicles are, for example (but not by way of limitation), water, saline, dextrose, glycerol, ethanol, or the like, and combinations thereof. In addition, the vehicle can contain minor amounts of auxiliary substances such as (but not limited to) wetting or emulsifying agents, biocompatible solvents, pH buffering agents, or adjuvants. The compositions of the present disclosure can also include ancillary substances, such as (but not limited to) pharmacological agents, cytokines, or other biological response modifiers.

The active agent can be delivered alone or as pharmaceutical compositions by any means known in the art, such as (but not limited to) systemically, regionally, or locally; by intra-arterial, intrathecal (IT), intravenous (IV), parenteral, intra-pleural cavity, topical, oral, transdermal delivery, or local administration, as subcutaneous, intra-tracheal (e.g., by aerosol) or transmucosal (e.g., buccal, bladder, vaginal, uterine, rectal, nasal mucosa), subcutaneous (SC), intracranial, intraocular, intracerebral, intracavitary, intraperitoneal, intranasal, intralymphatic, or intramuscular. Administration can also be localized directly into a tumor. Administration into the systemic circulation by intravenous or subcutaneous administration is typical. Intravenous administration can be, for example (but not by way of limitation), by infusion over a period such as (but not limited to) 30-90 min or by a single bolus injection.

Furthermore, the compositions can be formulated into compositions in either neutral or salt forms. Pharmaceutically acceptable salts include (but are not limited to) the acid addition salts (formed with the free amino groups of the active polypeptides) and which are formed with inorganic acids such as, for example, hydrochloric or phosphoric acids, or organic acids such as acetic, oxalic, tartaric, mandelic, and the like. Salts formed from free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, and procaine.

Compositions for therapies can be administered in a single dose treatment or in multiple dose treatments on a schedule and over a time period appropriate to the age, weight, and condition of the subject, the particular composition used, and the route of administration. In one non-limiting embodiment, a single dose of the composition according to the disclosure is administered. In other non-limiting embodiments, multiple doses are administered. The frequency of administration can vary depending on any of a variety of factors, e.g., severity of the symptoms, or whether the composition is used for prophylactic or curative purposes. For example, in certain non-limiting embodiments, the composition is administered once per month, twice per month, three times per month, every other week, once per week, twice per week, three times per week, four times per week, five times per week, six times per week, every other day, daily, twice a day, or three times a day. The duration of treatment (i.e., the period of time over which the composition is administered) can vary, depending on any of a variety of factors, e.g., subject response. For example, the composition can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

The compositions can be combined with a pharmaceutically acceptable carrier (excipient) to form a pharmacological composition. Pharmaceutically acceptable carriers can contain a physiologically acceptable compound that acts to, for example but not by way of limitation) stabilize or increase or decrease the absorption or clearance rates of the pharmaceutical compositions. Physiologically acceptable compounds can include, for example but not by way of limitation: carbohydrates, such as glucose, sucrose, or dextrans; antioxidants, such as ascorbic acid or glutathione; chelating agents; low molecular weight proteins; detergents; liposomal carriers; excipients; or other stabilizers and/or buffers. Other physiologically acceptable compounds include (but are not limited to) wetting agents, emulsifying agents, dispersing agents, or preservatives.

When administered orally, the present compositions may be protected from digestion. This can be accomplished either by combining the active agent with a composition to render it resistant to acidic and enzymatic hydrolysis or by packaging the active agent in an appropriately resistant carrier such as (but not limited to) a liposome, e.g., such as shown in U.S. Pat. No. 5,391,377.

For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. For topical transdermal administration, the agents are formulated into ointments, creams, salves, powders, and gels. Transdermal delivery systems can also include (for example but not by way of limitation) patches. The present compositions can also be administered in sustained delivery or sustained release mechanisms. For example, biodegradable microspheres or capsules or other biodegradable polymer configurations capable of sustained delivery of a peptide can be included herein.

For inhalation, the present compositions can be delivered using any system known in the art, including (but not limited to) dry powder aerosols, liquids delivery systems, air jet nebulizers, propellant systems, and the like. For example (but not by way of limitation), the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include (for example but not by way of limitation) air jet nebulizers.

In one aspect, the compositions are prepared with carriers that will protect the active agent against rapid elimination from the body, such as (but not limited to) a controlled release formulation, including implants and microencapsulated delivery systems. Biodegradable, biocompatible polymers can be used, such as (but not limited to) ethylene vinyl acetate, polyanhydrides, polyglycolic acid, collagen, polyorthoesters, and polylactic acid. Methods for preparation of such formulations will be apparent to those skilled in the art.

The active agent in general may be formulated to obtain compositions that include one or more pharmaceutically suitable excipients, surfactants, polyols, buffers, salts, amino acids, or additional ingredients, or some combination of these. This can be accomplished by known methods to prepare pharmaceutically useful dosages, whereby the active compound is combined in a mixture with one or more pharmaceutically suitable excipients. Sterile phosphate-buffered saline is one non-limiting example of a pharmaceutically suitable excipient.

In parenteral administration, the compositions will be formulated in a unit dosage injectable form such as (but not limited to) a solution, suspension, or emulsion, in association with a pharmaceutically acceptable excipient. Such excipients are inherently nontoxic and nontherapeutic. Non-limiting examples of such excipients include saline, Ringer's solution, dextrose solution, and Hanks' solution. Nonaqueous excipients such as (but not limited to) fixed oils and ethyl oleate may also be used. An alternative non-limiting excipient is 5% dextrose in saline. The excipient may contain minor amounts of additives such as (but not limited to) substances that enhance isotonicity and chemical stability, including buffers and preservatives.

Formulated compositions comprising the active agent can be used (for example but not by way of limitation) for subcutaneous, intramuscular, or transdermal administration. Compositions can be presented in unit dosage form, e.g., in ampoules or in multi-dose containers, with an added preservative. Compositions can also take such forms as suspensions, solutions, or emulsions in oily or aqueous vehicles, and can contain formulatory agents such as (but not limited to) suspending, stabilizing, and/or dispersing agents. Alternatively, the compositions can be in powder form for constitution with a suitable vehicle, e.g., sterile pyrogen-free water, before use.

The compositions may be administered in solution. The formulation thereof may be in a solution having a suitable pharmaceutically acceptable buffer, such as (but not limited to) phosphate, Tris (hydroxymethyl) aminomethane-HCl, or citrate, and the like. Buffer concentrations should be in the range of 1 to 100 mM. The formulated solution may also contain a salt, such as (but not limited to) sodium chloride or potassium chloride in a concentration of 50 to 150 mM. An effective amount of a stabilizing agent such as (but not limited to) mannitol, trehalose, sorbitol, glycerol, albumin, a globulin, a detergent, a gelatin, a protamine, or a salt of protamine may also be included.

Exemplary, non-limiting ranges for a therapeutically or prophylactically effective amount of the active agent, include a range of from about 0.001 mg/kg of the subject's body weight to about 500 mg/kg of the subject's body weight, such as but not limited to a range of from about 0.01 mg/kg to about 250 mg/kg, a range of from about 0.1 mg/kg to about 100 mg/kg, a range of from about 0.1 mg/kg to about 50 mg/kg, a range of from about 1 mg/kg to about 30 mg/kg, a range of from about 1 mg/kg to about 25 mg/kg, a range of from about 2 mg/kg to about 30 mg/kg, a range of from about 2 mg/kg to about 20 mg/kg, a range of from about 2 mg/kg to about 15 mg/kg, a range of from about 2 mg/kg to about 12 mg/kg, a range of from about 2 mg/kg to about 10 mg/kg, a range of from about 3 mg/kg to about 30 mg/kg, a range of from about 3 mg/kg to about 20 mg/kg, a range of from about 3 mg/kg to about 15 mg/kg, a range of from about 3 mg/kg to about 12 mg/kg, or a range of from about 3 mg/kg to about 10 mg/kg, or a range of from about 10 mg to about 1500 mg as a fixed dosage.

The composition is formulated to contain an effective amount of the active agent, wherein the amount depends on the subject to be treated and the severity of the condition of the subject. In certain non-limiting embodiments, the active agents may be administered at a dose ranging from about 0.001 mg to about 10 g, from about 0.01 mg to about 10 g, from about 0.1 mg to about 10 g, from about 1 mg to about 10 g, from about 1 mg to about 9 g, from about 1 mg to about 8 g, from about 1 mg to about 7 g, from about 1 mg to about 6 g, from about 1 mg to about 5 g, from about 10 mg to about 10 g, from about 50 mg to about 5 g, from about 50 mg to about 5 g, from about 50 mg to about 2 g, from about 0.05 μg to about 1.5 mg, from about 10 μg to about 1 mg protein, from about 30 μg to about 500 μg, from about 40 μg to about 300 μg, from about 0.1 μg to about 200 mg, from about 0.1 μg to about 5 μg, from about 5 μg to about 10 μg, from about 10 μg to about 25 μg, from about 25 μg to about 50 μg, from about 50 μg to about 100 μg, from about 100 μg to about 500 μg, from about 500 μg to about 1 mg, or from about 1 mg to about 2 mg. The specific dose level for any particular subject depends upon a variety of factors, including (but not limited to) the activity of the specific active agent, the age, body weight, general health, sex, diet, time of administration, route of administration, and rate of excretion, the drug combination, and the severity of the disease in the subject undergoing therapy.

The dosage of an administered active agent for humans will vary depending upon factors such as (but not limited to) the patient's age, weight, height, sex, general medical condition, and previous medical history. In certain non-limiting embodiments, the recipient is provided with a dosage of the active agent(s) that is in the range of from about 1 mg to about 1000 mg as a single infusion or single or multiple injections, although a lower or higher dosage also may be administered. In certain non-limiting embodiments, the dosage may be in the range of from about 25 mg to about 100 mg per square meter (m²) of body surface area for a typical adult, although a lower or higher dosage also may be administered. Non-limiting examples of dosages that may be administered to a human subject further include 1 to 500 mg, 1 to 70 mg, or 1 to 20 mg, although higher or lower doses may be used. Dosages may be repeated as needed, for example (but not by way of limitation), once per week for 4-10 weeks, once per week for 8 weeks, or once per week for 4 weeks. It may also be given less frequently, such as (but not limited to) every other week for several months, or more frequently, such as twice weekly or by continuous infusion.

In some non-limiting embodiments, the amount of an active agent is in a concentration of about 1 nM, about 5 nM, about 10 nM, about 25 nM, about 50 nM, about 75 nM, about 100 nM, about 150 nM, about 200 nM, about 250 nM, about 300 nM, about 350 nM, about 400 nM, about 500 nM, about 550 nM, about 600 nM, about 700 nM, about 800 nM, about 900 nM, about 1 μM, about 2 μM, about 3 μM, about 4 μM, about 5 μM, about 6 μM, about 7 μM, about 8 μM, about 9 μM, about 10 μM, about 15 μM, about 20 μM, about 25 μM, about 30 μM, about 35 μM, about 40 μM, about 45 μM, about 50 μM, about 60 μM, about 70 μM, about 75 μM, about 80 μM, about 90 μM, about 100 μM, about 125 μM, about 150 μM, about 175 μM, about 200 μM, about 250 μM, about 300 μM, about 350 μM, about 400 μM, about 500 μM, about 600 μM, about 700 μM, about 750 μM, about 800 μM, about 900 μM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 100 mM, about 110 mM, about 120 mM, about 130 mM, about 140 mM, about 150 mM, about 160 mM, about 170 mM, about 180 mM, about 190 mM, about 200 mM, about 250 mM, about 300 mM, about 400 mM, about 500 mM, about 600 mM, about 700 mM, about 800 mM, about 900 mM, about 1000 mM, about 1 M, about 1.1 M, about 1.2 M, about 1.3 M, about 1.4 M, about 1.5 M, about 1.6 M, about 1.7 M, about 1.8 M, about 1.9 M, about 2 M, about 3 M, about 4 M, about 5 M, about 6 M, about 7 M, about 8 M, about 9 M, about 10 M, about 15 M, about 20 M, about 25 M, about 30 M, about 35 M, about 40 M, about 45 M, about 50 M, about 75 M, about 100 M, or any range in between any two of the aforementioned concentrations, including said two concentrations as endpoints of the range, or any number in between any two of the aforementioned concentrations.

The number of dosages administered depends on the severity of the condition and the response to therapy (e.g., whether presenting acute or chronic symptoms) Treatment can be repeated for recurrence of an acute disorder or acute exacerbation. For chronic disorders, the active agent can be administered at regular intervals, such as (but not limited to) weekly, fortnightly, monthly, quarterly, every six months for at least 1, 5, or 10 years, or for the life of the patient if the condition is chronic.

In certain non-limiting embodiments, pharmaceutical compositions for parenteral administration are sterile, substantially isotonic, and manufactured under GMP conditions. Pharmaceutical compositions can be provided in unit dosage form (i.e., the dosage for a single administration). Pharmaceutical compositions can be formulated using one or more physiologically acceptable carriers, diluents, excipients, or auxiliaries. The formulation depends on the route of administration chosen. For injection, the active agent can be formulated in aqueous solutions, such as (but not limited to) in physiologically compatible buffers such as Hank's solution, Ringer's solution, or physiological saline or acetate buffer (to reduce discomfort at the site of injection). The solution can contain formulatory agents such as (but not limited to) suspending, stabilizing, and/or dispersing agents. Alternatively, the active agent can be in lyophilized form for constitution with a suitable vehicle, e.g., sterile pyrogen-free water, before use.

Some non-limiting embodiments provided herein include kits. In some non-limiting embodiments, a kit can include a quantity of an active agent as described or otherwise contemplated herein. In some non-limiting embodiments, the active agent is lyophilized. In some non-limiting embodiments, the active agent is in aqueous solution, or other carrier as described herein. In some non-limiting embodiments, the kit includes a pharmaceutical carrier for administration of the active agent. Certain non-limiting embodiments of the present disclosure include kits containing components suitable for treatments or diagnosis. Exemplary kits may contain at least one active agent. A device capable of delivering the kit components by injection, for example, a syringe for subcutaneous injection, may be included in some non-limiting embodiments. Where transdermal administration is used, a delivery device such as hollow microneedle delivery device may be included in the kit in some non-limiting embodiments. Exemplary transdermal delivery devices are known in the art, such as (but not limited to) a hollow Microstructured Transdermal System (e.g., 3M Corp.), and any such known device may be used. The kit components may be packaged together or separated into two or more containers. In some non-limiting embodiments, the containers may be vials that contain sterile, lyophilized formulations of a composition that are suitable for reconstitution. A kit may also contain one or more buffers suitable for reconstitution and/or dilution of other reagents. Alternatively, the active agent may be delivered and stored as a liquid formulation. Other containers that may be used include, but are not limited to, a pouch, tray, box, tube, or the like. Kit components may be packaged and maintained sterilely within the containers. Another component that can be included is instructions for the use of the kit for treatment.

The active agents of the present disclosure can be combined into formulations or treatments that are synergistic. As used herein the terms “synergism,” “synergistic,” or “synergistic effect” refers to a therapeutic effect or result that is greater than the additive effects of each active agent used individually. Presence or absence of a synergistic effect for a particular combination of treatment substances can be quantified by using the Combination Index (CI) (e.g., Chou, Pharmacol Rev, 2006. 58(3): 621-81), wherein CI values lower than 1 indicate synergy and values greater than 1 imply antagonism. Combinations of the inhibitors and antagonists of the present disclosure can be tested in vitro for synergistic cell growth inhibition using standard cell lines for particular cancers, or in vivo using standard animal cancer models. A synergistic effect of a combination described herein can permit, in some embodiments, the use of lower dosages of one or more of the components of the combination. A synergistic effect can also permit, in some embodiments, less frequent administration of at least one of the administered active agents. Such lower dosages and reduced frequency of administration can reduce the toxicity associated with the administration of at least one of the therapies to a subject without reducing the efficacy of the treatment.

The term “coadministration” refers to administration of two or more active agents, e.g., a heparanase inhibitor and an anticancer drug. The timing of coadministration depends in part of the combination and compositions administered and can include administration at the same time, just prior to, or just after the administration of one or more additional therapies Coadministration is meant to include simultaneous or sequential administration of the compound and/or composition individually or in combination. Thus, the preparations can also be combined, when desired, with other active substances (e.g., to reduce metabolic degradation). For example, the compositions described herein can be used in combination with one another, or with other active agents known to be useful in treating cancer.

In particular, non-limiting examples, the active agent of the present disclosure can be combined with liposomes in which another cargo molecule, e.g., an anticancer drug, is disposed. In addition to other pharmaceutically acceptable carrier(s), the liposome may contain amphipathic agents such as lipids which exist in an aggregated form as micelles, insoluble monolayers, liquid crystals, or lamellar layers in aqueous solution. Suitable lipids for liposomal formulation include, but are not limited to, monoglycerides, diglycerides, sulfatides, lysolecithin, phospholipids, saponin, bile acids, combinations thereof, and the like. Preparation of such liposomal formulations is well within the level of ordinary skill in the art, as disclosed, for example, in U.S. Pat. Nos. 4,235,871; 4,501,728; 4,837,028; and 4,737,323; the entire contents of each of which are incorporated herein by reference. As used herein, the term “liposome” means a vesicle composed of amphiphilic lipids arranged in a spherical bilayer or bilayers. Liposomes are unilamellar or multilamellar vesicles which have a membrane formed from a lipophilic material and an aqueous interior. The aqueous portion may contain the active agent to be delivered. Liposomes can be made from phospholipids other than naturally-derived phosphatidylcholine. Neutral liposome compositions, for example, can be formed from dimyristoyl phosphatidylcholine (DMPC) or dipalmitoyl phosphatidylcholine (DPPC) or other similar lipids. Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example (but not by way of limitation), soybean PC, and egg PC. Another type is formed from mixtures of phospholipid and/or phosphatidylcholine and/or cholesterol.

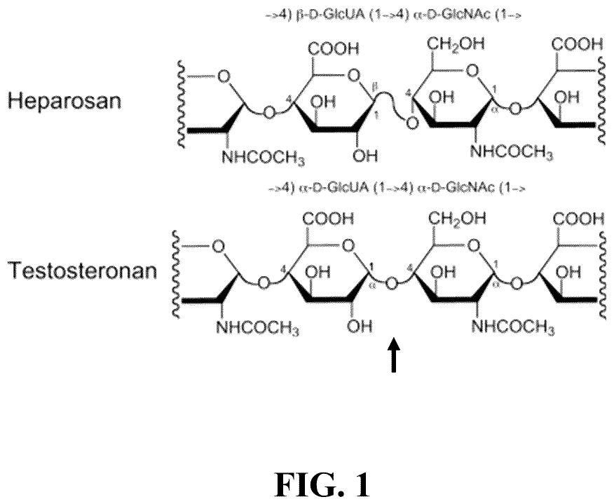

Returning the discussion of various particular embodiments of the present disclosure, as previously noted, sTestan is a heparosan (HEP) analog synthesized by a microbial GAG synthase, the enzyme CtTS from the opportunistic pathogen Comamonas testosteroni, which produces the polysaccharide Testan [-4-D-GlcUA-α1,4-D-GlcNAc-α1-]ₙ. Testan comprises the same sugars as the biomedically relevant GAGs HEP (or N-acetyl-heparosan), but there is a difference in the linkage (the glycosidic bond) between the GlcUA-GlcNAc in the co-polymer chains. In Testan, the GlcUA-GlcNAc are alpha-linked, while in HEP, it is beta-linked (FIG. 1). The other linkage between the GlcNAc-GlcUA sites in the co-polymer remains the same, namely alpha-linked. It is widely known that the nature of the glycosidic linkages in carbohydrates have important implications for their bioactivity. For example, at the molecular level cellulose (wood and paper) and starch (e.g., bread) are both 1→4-Glucose polymers with either beta- or alpha-links, respectively. Therefore, Testan and its derivatives will have distinct properties useful for medical applications. Structures and methods of synthesis of Testan are shown in U.S. Pat. Nos. 9,695,427 and 10,273,517, each of which is expressly incorporated herein by reference in its entirety.

Certain non-limiting embodiments of the present disclosure include a polymer comprising the repeat structure [4-D-glucuronic acid-α1,4-D-N-acetylglucosamine-α1-]ₙ ([-4-D-GlcUA-α1,4-D-GlcNAc-α1-]ₙ), wherein at least one sulfur moiety is linked to the repeat structure, and wherein n is in a range of from about 2 to about 500. For example, but not by way of limitation, n may be about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 55, about 60, about 65, about 70, about 75, about 80, about 85, about 90, about 95, about 100, about 110, about 120, about 130, about 140, about 150, about 160, about 170, about 180, about 190, about 200, about 225, about 250, about 275, about 300, about 325, about 350, about 375, about 400, about 425, about 450, about 475, or about 500. In addition, the scope of the present disclosure explicitly includes n values in a range formed from any two of the above values (e.g., a range of from about 2 to about 500, a range of from about 2 to about 200, a range of from about 10 to about 100, a range of from about 10 to about 300, etc.).

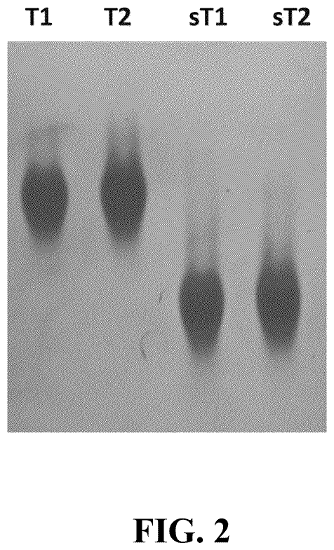

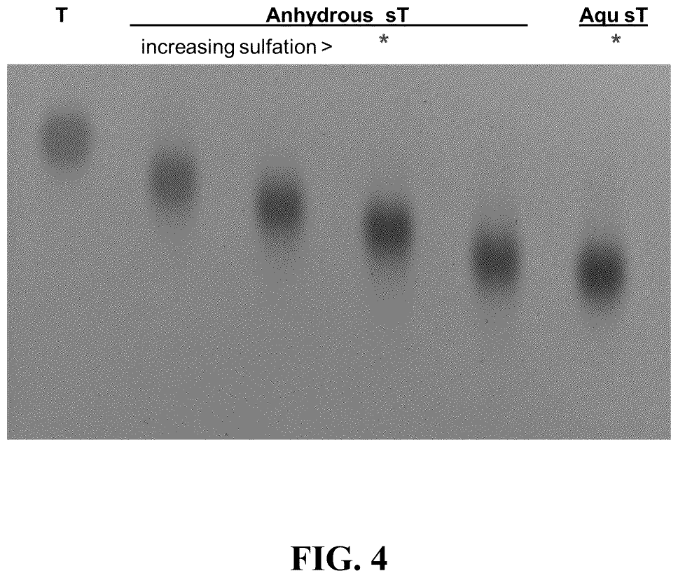

In one non-limiting embodiment of the present disclosure, sTestan forms the basis for a new class of competitive inhibitors of heparanase, which is noted above as the catabolic enzyme that processes heparan sulfate (HS) in animals, and which is an important therapeutic target for cancer treatment (18-19). As one example of chemical sulfation of sTestan, sulfur trioxide-trimethyl amine complex in basic aqueous (aq) solution afforded an O-sulfated-Testan (FIG. 2). In another example, the sulfur trioxide-trimethyl amine complex is employed in an anhydrous (anh; dry) aprotic solvent (e.g., dimethylformamide, formamide) (FIG. 4). O-sulfated heparosan-based polymers have the modification that is recognized by human heparanase (13).

As used herein in reference to sTestan, the term “sulfate” refers to the moiety comprising an oxygen of GlcNAc or, alternatively, of GlcUA, linked to an SO₃ group, i.e., R—O—SO₃ where R=monosaccharide. In other words, the monosaccharide's hydroxyl is sulfonated such that a sulfate group is introduced. Therefore, as used herein in reference to sTestan having O-linked sulfur, the terms “sulfated” and “sulfonated” are interchangeable.

In certain embodiments, the active agent of the present disclosure is an sTestan linked to a heparosan chain to form an sTestan-heparosan chimera. For example (but not by way of limitation), the sTestan portion is the actual inhibitory moiety, while the heparosan portion is used to assist or direct binding to the heparanase target or to alter pharmacokinetic behavior (e.g., extend half-life, control tissue access, etc.).

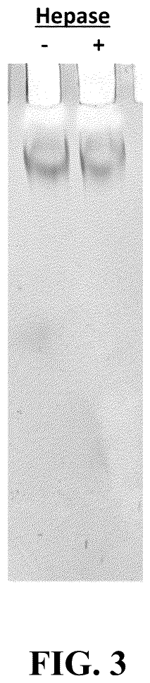

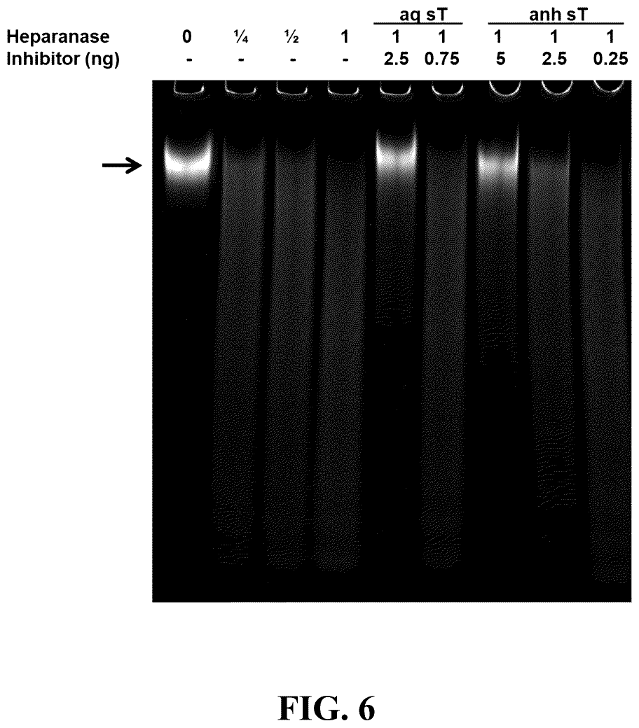

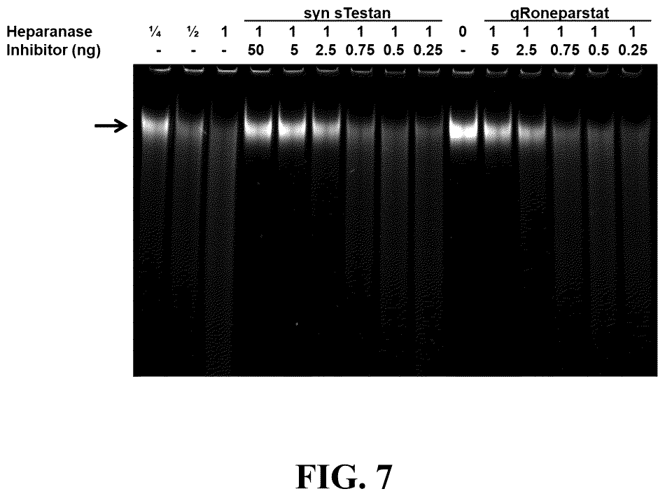

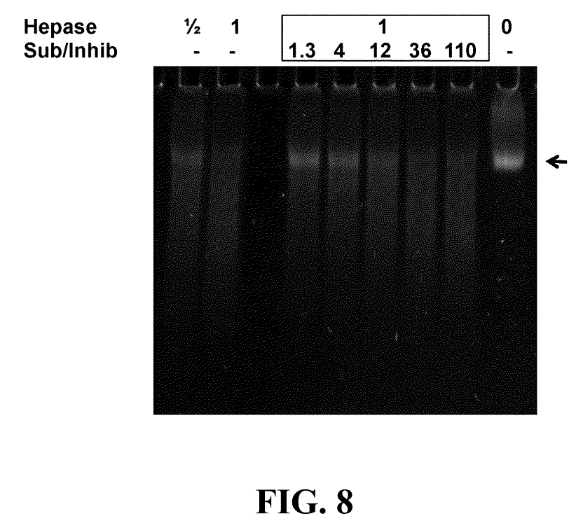

Recombinant human heparanase cleaves O-sulfo heparosan and natural HS species, but not the sTestan (FIG. 3). Furthermore, co-incubation of sTestan with the O-sulfo heparosan substrate inhibited the digestion of the substrate (FIGS. 6 and 7). Similarly, sTestan acted as an inhibitor, protecting natural HS from human heparanase digestion (FIG. 8) and thereby demonstrating that this novel polymer acts as a competitive inhibitor against heparanase and thus has oncology applications. sTestan was at least ˜100-fold more active on a molar basis than the heparosan substrate, based on its IC₅₀ in the 5-20 nM range. This finding indicates tighter heparanase binding to the sTestan, with alpha-alpha linkages than polysaccharides with only natural alternating beta-alpha linkages of the HS family. Further, in FIG. 7, sTestan was shown to be a potent competitive inhibitor of human heparanase similar to a known heparanase inhibitor, generic Roneparstat (U.S. Pat. No. 7,781,416).

With respect to the higher-than-expected levels of human heparanase inhibition exhibited by the sTestan polymers, and without wishing to be bound by theory, it was predicted that the alpha-linkage bonds mimic some aspects of the transition state of the heparan sulfate chain undergoing cleavage in the heparanase active site (13). The glycosidic bond emerging from GlcUA residue in sTestan occupies an axial position rather than the typical equatorial position found in the natural HS chains. Therefore, these polymers at the heparanase target scissile bond could align with the catalytic nucleophile in the enzyme active site as found in the canonical retaining glycosidase mechanism (12). However, the enzyme is unable to cleave the chain, which is bound tightly, and thus denies other HS chains access to the active site, thereby acting as a competitive enzyme inhibitor.

It is well known that transition state analogs are better inhibitors than strict substrate structural analogs with unperturbed bonds (12). Thus, in comparison to other types of heparanase inhibitors used heretofore in the clinic (10, 14), the sTestan polymers disclosed herein represent a new class of therapeutic drugs.

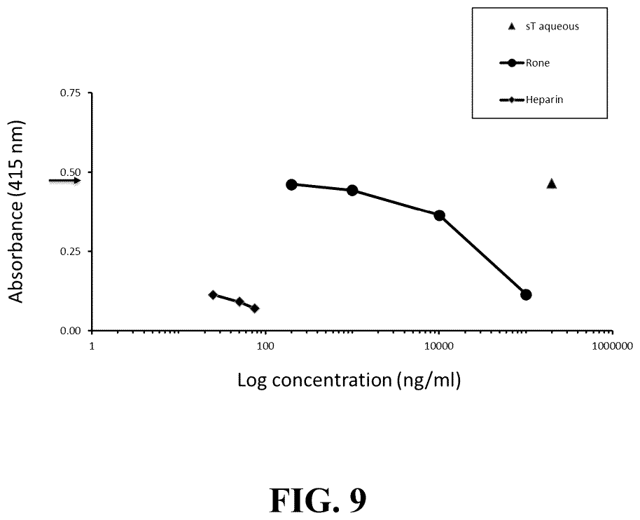

In addition, the sTestan polysaccharide structures may either enhance or decrease interactions with various GAG-binding proteins, depending on the protein and polysaccharide studied. sTestan therefore comprises enhanced selectivity in comparison to natural HS-based sugars, which bind to a plethora of proteins (e.g., heparin binds to over 200 different proteins in human plasma), thereby resulting in a multitude of useful biological activities. For example, if only a subset of the proteins in the ‘heparinome’ interact with a sTestan family polymer, then selectivity is possible, allowing the potential for side effect reduction in the patient. In one example, sTestan was shown to not be an anticoagulant; sTestan is at least ˜20,000-fold less effective than heparin (FIG. 9), therefore thrombin/antithrombin III interaction is not affected by sTestan. Further, sTestan, when used as a drug in the human body, should not have the side effect of excess bleeding and thus will be a safer therapeutic.

The parameters of (i) polymer chain size and (ii) sulfation level and positional pattern can be modified to suit the desired bioactivity of the sTestan class molecules. The utility of sTestan promises to expand the possibilities for selective and improved therapeutics.

### EXAMPLE

Examples are provided hereinbelow. However, the present disclosure is to be understood to not be limited in its application to the specific experimentation, results, and laboratory procedures disclosed herein after. Rather, the Examples are simply provided as one of various embodiments and are meant to be exemplary, not exhaustive.

All reagents were purchased from Sigma-Aldrich or other commercial vendors unless otherwise noted and were used without further purification. Any source of or method of producing the Testan backbone polymer is suitable, including for example, employing (i) native or recombinant microbes with the CtTs gene or active mutants or derivatives in vivo, or (ii) extracts with recombinant microbes with the CtTs enzyme or active mutants or derivatives in vitro with the correct precursors under the appropriate conditions (e.g., UDP-sugars, divalent cation-containing reaction buffer, etc.), see, for example, Otto, op. cit.; and U.S. Pat. Nos. 9,695,427, and 10,273,517.

For example, in one non-limiting embodiment, the Testan backbone polymer was produced by the fermentation of the native bacteria, C. testosteroni KF-1, in chemically defined media as described in Otto, N. J., Solakyildirim, K., Linhardt, R. J., DeAngelis, P. L. Comamonas testosteronan synthase, a bifunctional glycosyltransferase that produces a unique heparosan polysaccharide analog. Glycobiology. 21(10):1331-1340 (2011). However, the source of the starting backbone is not limiting for use in producing the compounds of the present disclosure.

### Polysaccharide Quantitation.

The amount of polymers was measured by the carbazole assay with a GlcUA standard (13); every other sugar residue in sTestan-based chains is GlcUA.

### Polysaccharide Sulfation

Various methods for Testan sulfation and sulfonation, using either a chemical reagent (e.g., sulfur trioxide complexes, chlorosulfonic acid) or recombinant GAG sulfotransferase enzymes in vivo or in vitro can be employed. Non-limiting examples of such methods are shown below.

Synthesis of sTestan Using a Chemical Route:

Various sulfation methods can be employed (16,17). In one non-limiting example, to create a sTestan prototype, a solution of Testan polymer dissolved in water was adjusted with concentrated NaOH to achieve 0.05-4 M NaOH and 1-15 mg/ml carbohydrate final concentrations. Then the sulfation reagent (e.g., solid sulfur trioxide: trimethylamine complex; Aldrich) was added to the reaction in a desired ratio (e.g., a ratio of 0.1:1 to 100:1 reagent/polymer w/w) with mixing. The suspension was rotated or inverted at a temperature in a range of from 2° C. to 40° C. for a period in a range of from 10 minutes to 2 days. Any remaining solid sulfation reagent was then removed by centrifugation (e.g., 1,000-20,000×g, 1-10 min), and the solution was neutralized with HCl with mixing. The material was then subjected to 6-7 rounds of ultrafiltration against water using a spin unit (e.g., 3 kDa MWCO), wherein the resulting concentrate with the sTestan was then harvested. Further polishing by strong anion exchange chromatography with a NaCl elution (e.g., Sepharose Q) and/or reverse phase extraction (e.g., C18 resin, solvent, etc.) is used to increase the purity level and/or remove undesirable contaminants.

As an alternative solvent system with the same sulfur trioxide: trimethylamine complex sulfation reagent or other sulfation reagents (e.g., chlorosulfonic acid), dry (with 3 Angstrom molecular sieves) formamide was used to dissolve lyophilized (freeze-dried) Testan. The solution was reacted with the sulfation reagent at various ratios, then incubated at 0-40° C. for 0.1-48 hrs. For anhydrous sulfation reactions, purification was achieved by a similar process as for aqueous reactions; in some cases, alcohol precipitation (e.g., 2.5-4 volumes of ethanol or isopropanol, 20-120 min, −20 to 22° C., followed by harvesting by centrifugation) was performed prior to ultrafiltration or chromatography to remove the reaction solvent.

Synthesis of sTestan Using an Enzymatic Sulfation Route:

Enzymes which are able to transform heparosan into heparan sulfate can also be used to transform Testan into sTestan (see Li, G., Masuko, S., Green, D. E., Xu, Y., Li, L., Zhang, F., Xue, C., Liu, J., DeAngelis, P. L., Linhardt, R. J. N-sulfotestosteronan, a novel substrate for heparan sulfate 6-O-sulfotransferases and its analysis by oxidative degradation. Biopolymers. 99(10):675-685 (2013)). However, as there are many sulfo-transferases, different catalysts can be employed as long as the sulfation pattern and level yields sTestan with the desired bioactivity or inhibitor activity.

Overall, the use of controlled microbial (native or recombinant) fermentation in vivo or chemoenzymatic synthesis in vitro combined with chemical or enzymatic sulfation steps yielded more defined and reproducible products than the current heparin-derived products. Furthermore, the envisioned sTestan supply chain is more secure and more tamper-proof than animal sourcing. Compared to current small molecular weight compounds made by organic synthesis, the various pathways for sTestan synthesis are more ‘green,’ with less toxic reagents and less hazardous byproducts.

### Analysis of Sulfated Polysaccharides

Agarose gel (1-1.5%, 1×TAE) or polyacrylamide gel electrophoresis (PAGE; 6-20% gel, 1×TBE) analyses were used to analyze the conversion of Testan into sTestan. The polymers were stained with Stains-All dye to visualize the bands; this dye also yields charge density information. Testan stains as a slower migrating band with a blue color, while sTestan runs as a faster band with a purple color (FIG. 2); the latter polymer has a higher charge density due to the sulfate addition thus the molecule both migrates faster and has a shifted color output.

FIG. 4 contains another gel analysis of Testan (T) and various sulfated Testans (sT) produced using sulfation level control (using reagent stoichiometry) and regio-specific control (solvent system). The color change observed from blue to purple to yellow/orange indicates increased sulfation levels. The precise sulfation position can shift the hydrodynamic radius of the Testan depending on the position(s) on the sugar ring that is modified, even if the overall density of sulfate is equivalent. Therefore, the aq and anh sTestan polymers have altered migration speed even at similar sulfation levels.

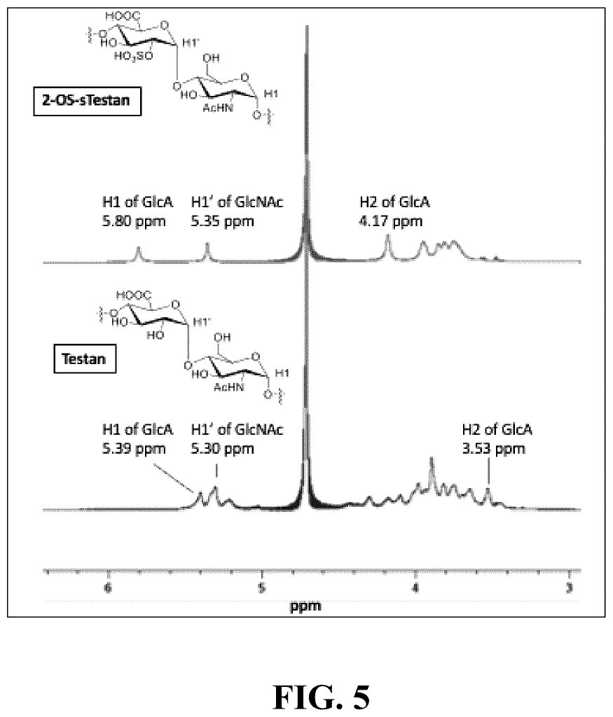

The aqueous sulfated Testan was also examined by NMR (FIG. 5) and LC-MS. 1D and 2D NMR and LC-MS indicated that the 2-OH of the GlcA sugar was highly modified under the conditions, forming “aq sTestan.” This finding was unexpected, as the 6-OH of GlcNAc, as a primary hydroxyl functionality, was typically the most sulfated position in heparosan and HS derivatives when using the same sulfur trioxide complex in organic solvents.

### Heparanase Challenge

Recombinant human heparanase (73 ng; R&D Systems; Minneapolis, MN) was incubated for 24 hours with sTestan polysaccharide (180 ng) in 50 mM sodium acetate, pH 5, 1 mg/ml acetylated bovine serum albumin (Promega; Madison, WI) at 30° C. The reactions were analyzed by polyacrylamide gel electrophoresis (20% gels, 1×TBE) and Stains-All detection. As can be seen in FIG. 3, recombinant human heparanase cleaves O-sulfo heparosan and natural HS species, but not the sTestan.

### Heparanase Enzyme Inhibition Assays

A gel-based assay was used to monitor the catalytic action of recombinant human heparanase (R&D Systems; Minneapolis, MN) on polysaccharide substrates. In these assays, fluorescent chains were monitored for cleavage; the disappearance of the parental substrate band is readily noticeable. Each chain contains multiple, overlapping heparanase cut sites so there is a gradual decrease in molecular weight over time. This simple, defined assay directly monitors the digestion of polysaccharides (i.e., glycans>˜20 monosaccharide units) that approximate naturally occurring substrates.

Two types of substrates were employed to follow heparanase activity: (i) a fluorescein end-labeled synthetic O-sulfated O-linked heparosan or (ii) polyacrylamide gel electrophoretically-purified rhodamine-tagged HS. Heparanase (1.3 ng/μl final) was incubated with substrate (synthetic, 57 ng/μl; HS, 3.3 ng/μl) for various times (typically ˜0.2-5 h) in 50 mM sodium acetate, pH 5, 1 mg/ml acetylated bovine serum albumin (Promega; Madison, WI) at 30° C. Aliquots of reactions were analyzed by polyacrylamide gel electrophoresis (2 μl of reaction/lane; 20% gels, 1×TBE) and fluorescence imaging (5-120 sec exposures; ChemiDoc MP). Then the overall size range of the polysaccharide fragments was followed by Stains-All detection. For the inhibition studies, the sTestan preparation was titrated at various molar ratios to the fluorescent substrate. Parallel control digest reactions with 25% (¼) or 50% (½) of the enzyme were used to help calibrate the progress of reactions or level of inhibition and were used as a comparison to assess the IC₅₀ of the experimental samples.

Co-incubation of sTestan with the synthetic O-sulfo heparosan substrate inhibited the digestion of the substrate (FIG. 6). Similarly, sTestan acted as an inhibitor, protecting natural HS from human heparanase digestion (FIG. 8) and thereby demonstrating that this novel polymer acts as a competitive inhibitor against heparanase and thus has oncology applications. sTestan was at least ˜100-fold more active on a molar basis than the heparosan substrate, based on its IC₅₀ in the 5-20 nM range. This finding indicates tighter heparanase binding to the sTestan, with alpha-alpha linkages than polysaccharides with only natural alternating beta-alpha linkages of the HS family.

Further, sTestan was shown to be similarly potent as a competitive inhibitor of human heparanase to a known heparanase inhibitor (generic Roneparstat) (FIG. 7).

### Anticoagulant Assays

The Chromogenix Coatest assay (Diapharma; West Chester, OH) was used to measure the effect on thrombin (the clotting factor that converts fibrinogen into fibrin glue) inactivation by antithrombin III. Typically, heparin (here the international heparin standard) is required to effectively reduce the thrombin activity (from human plasma), as measured by absorbance (at 415 nm) of a chromogenic thrombin substrate. The aq sTestan polymer was tested at various concentrations compared to a heparin standard curve; as can be seen in FIG. 9, sTestan was not an anticoagulant.

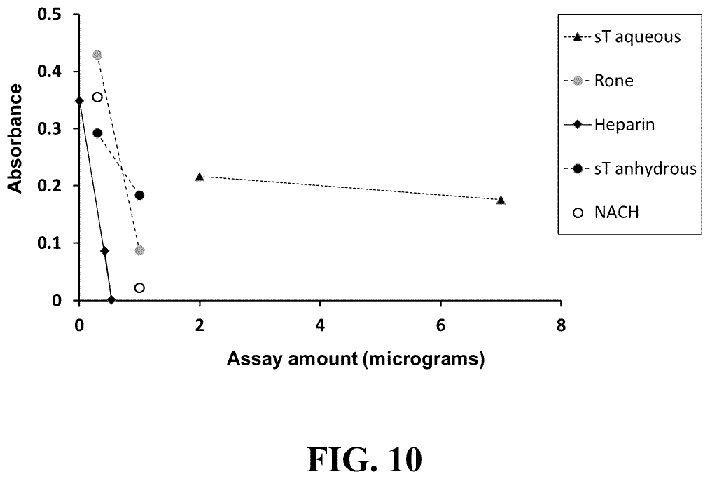

A modification of a diagnostic HIT assay (Zymutest HIA IgG; Aniara Diagnostica, West Chester, OH) was used to evaluate sTestan for side effects. A titration of sTestan was used to compete with the immobilized heparin for the HIT complex IgG binding site found in the assay positive control. ELISA methodology was used to measure the bound antibody as measured by absorbance (at 415 nm) of the product derived from a chromogenic substrate by the enzyme on the secondary antibody probe. Free heparin competes for plate-bound heparin, reducing the absorbance compared to the control with a free sugar polymer. Heparin derivatives treated to reduce their anticoagulant activity (non-anticoagulant heparin (NACH) or Roneparstat (Rone)) were also tested in the HIT assay. As shown in FIG. 10, sTestan (either aqueous or anhydrous sulfation) appeared to have less or similar HIT potential than heparin and its derivatives.

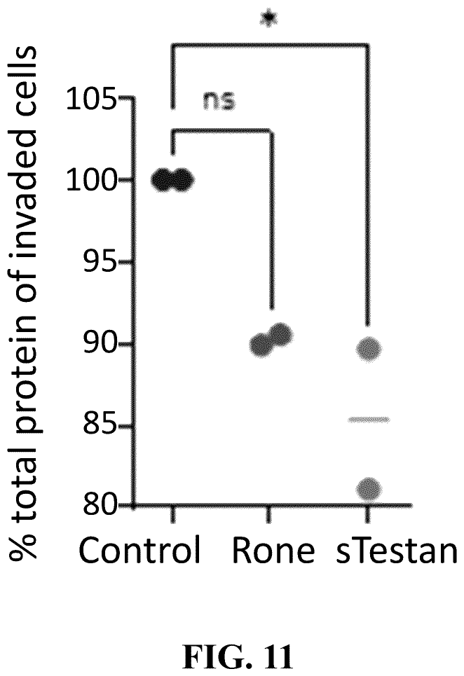

A trans-well metastasis assay was used to evaluate the efficacy of sTestan for oncological treatments. Human ovarian cancer cells (OV9; 1×10⁵ in 200 μl DMEM without serum) were seeded onto the top chamber of a Matrigel-coated Boyden transwell filter, alongside 20 μM sTestan variant or a known inhibitor (generic Roneparstat), or PBS control. The lower chamber contains 600 μl DMEM with 10% FBS. Plates were incubated at 37° C. for 24 h, then the cells were extracted and assayed for protein. The protein level in the lower compartment was used as a proxy for the invading cell number. As shown in FIG. 11, sTestan and Roneparstat had similar effects in suppressing metastasis.

sTestan-Based Chimeric Polymer Production

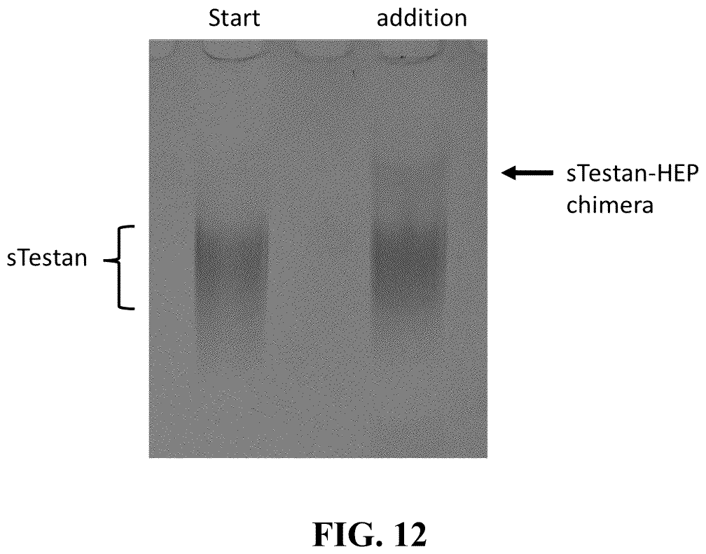

The sTestan polymers of the present disclosure may further benefit from being linked to a moiety (such as, but not limited to, a heparosan chain) to assist or direct binding to the heparanase target or to alter pharmacokinetic behavior (e.g., extend half-life, control tissue access, etc.). FIG. 12 demonstrates the production of a sTestan-heparosan chimera using the recombinant Pasteurella multocida synthase G (20) to co-polymerize the monosaccharides from both UDP-sugar donors onto the non-reducing end of the sTestan molecule (similar to the process in (15)). As shown in FIG. 12, the sTestan can be elongated with heparosan to create a chimeric polysaccharide, as shown by the larger molecular weight product. This new chimeric material will have different pharmacokinetics compared to the starting sTestan polymer alone.

### NON-LIMITING ILLUSTRATIVE EMBODIMENTS

Illustrative embodiment 1. A polymer, comprising the repeat structure [4-D-glucuronic acid-α1,4-D-N-acetylglucosamine-α1-]ₙ ([-4-D-GlcUA-α1,4-D-GlcNAc-α1-]ₙ), wherein at least one sulfur moiety is linked to at least one hydroxyl of the repeat structure, and wherein n is in a range of from about 2 to about 500.

Illustrative embodiment 2. The polymer of Illustrative embodiment 1, wherein the sulfur moiety is SO₃.

Illustrative embodiment 3. The polymer of Illustrative embodiment 1 or 2, comprising the structure O-Sulfo [-4-GlcUA-α1,4-GlcNAc-α1-]ₙ.

Illustrative embodiment 4. The polymer of any of Illustrative embodiments 1-3, wherein the sulfur moiety is O-linked to the GlcNAc.

Illustrative embodiment 5. The polymer of any of Illustrative embodiments 1-4, wherein the sulfur moiety is O-linked to the GlcUA.

Illustrative embodiment 6. A composition, comprising the polymer of any one of Illustrative embodiments 1-5.

Illustrative embodiment 7. The composition of Illustrative embodiment 6, wherein the composition is in an aqueous solution.

Illustrative embodiment 7A. The composition of Illustrative embodiment 6 or 7, further comprising at least one additional active agent (e.g., an anticancer drug or other active agent for co-administration with the polymer).

Illustrative embodiment 8. A composition, comprising: at least one polymer of any one of Illustrative embodiments 1-5; and a heparosan chain or other polymer linked thereto.

Illustrative embodiment 8A. The composition of any of Illustrative embodiments 6-8, further comprising at least one additional active agent (e.g., an anticancer drug or other active agent for co-administration with the polymer).

Illustrative embodiment 9. A method of inhibiting heparanase activity, comprising exposing the heparanase to at least one polymer of any one of Illustrative embodiments 1-5.

Illustrative embodiment 10. A method of inhibiting heparanase activity, comprising exposing the heparanase to at least one composition of any one of Illustrative embodiments 6-8.

Illustrative embodiment 11. The method of Illustrative embodiment 9 or 10, wherein the heparanase activity is inhibited in vitro.

Illustrative embodiment 12. The method of Illustrative embodiment 9 or 10, wherein the heparanase activity is inhibited in vivo.

Illustrative embodiment 13. A pharmaceutical composition, comprising: at least one polymer of any one of Illustrative embodiments 1-5; and a pharmaceutically acceptable excipient.

Illustrative embodiment 13A. The pharmaceutical composition of claim 13, further defined as a sterile pharmaceutical composition.

Illustrative embodiment 13B. The pharmaceutical composition of Illustrative embodiment 13 or 13A, further comprising at least one additional active agent (e.g., an anticancer drug or other active agent for co-administration with the polymer).

Illustrative embodiment 14. A method of treating a subject in need of treatment, comprising the step of: administering the pharmaceutical composition of Illustrative embodiment 13 to the subject in need of treatment.

Illustrative embodiment 15. The method of Illustrative embodiment 14, wherein the subject has cancer or is predisposed to cancer.

Illustrative embodiment 16. The method of Illustrative embodiment 14, wherein the subject has at least one disease or condition associated with overexpression, misregulation, or hyperactivity of heparanase.

Illustrative embodiment 17. The method of Illustrative embodiment 16, wherein the at least one disease or condition is selected from the group consisting of diabetes, a complication of diabetes (e.g., cardiomyopathy), atherosclerosis, thrombosis, a viral infection (e.g., herpes simplex), and combinations thereof.

Illustrative embodiment 17A. The method of any of Illustrative embodiments 14-17, further comprising the step of administering at least one additional active agent (e.g., an anticancer drug or other active agent for concomitant administration with the polymer) simultaneously or wholly or partially sequentially with the pharmaceutical composition.

Illustrative embodiment 18. A method of producing a sulfated testan polymer, the method comprising the steps of: producing a polymer backbone having the repeat structure [4-D-glucuronic acid-α1,4-D-N-acetylglucosamine-α1-]ₙ ([-4-D-GlcUA-α1,4-D-GlcNAc-α1-]ₙ), wherein n is in a range of from about 2 to about 500; and linking at least one sulfur moiety to at least one hydroxyl of the repeat structure.

Illustrative embodiment 19. The method of Illustrative embodiment 18, wherein the polymer backbone is produced by native fermentation or recombinant production using an organism expressing a testosteronan synthase gene.

Illustrative embodiment 20. The method of Illustrative embodiment 18, wherein the polymer backbone is produced by chemical or chemoenzymatic synthesis.

Illustrative embodiment 21. A kit, comprising at least one polymer of any one of Illustrative embodiments 1-5 and/or at least one composition of any one of Illustrative embodiments 6-8.

Illustrative embodiment 21. The kit of Illustrative embodiment 21, further comprising a device for delivering the polymer or composition to a subject.

Illustrative embodiment 22. A composition for use in the method of any one of Illustrative embodiments 9-12 and 14-17, wherein the composition comprises a polymer comprising the repeat structure [4-D-glucuronic acid-α1,4-D-N-acetylglucosamine-α1-]ₙ ([-4-D-GlcUA-α1,4-D-GlcNAc-α1-]ₙ), wherein at least one sulfur moiety is linked to at least one hydroxyl of the repeat structure, and wherein n is in a range of from about 2 to about 500.

Illustrative embodiment 23. Use of at least one polymer of any one of Illustrative embodiments 1-5 and/or at least one composition of any one of Illustrative embodiments 6-8 in a method of treating a subject in need of treatment.

### CITED REFERENCES

The following references, to the extent that they provide exemplary procedural or other details supplementary to those set forth herein, are specifically incorporated herein by reference. In addition, the following is not intended to be an Information Disclosure Statement; rather, an Information Disclosure Statement in accordance with the provisions of 37 CFR § 1.97 will be submitted separately.
1. Köwitsch, A., Zhou, G. & Groth, T., Medical application of glycosaminoglycans: a review. J. Tissue. Eng. Regen. Med. 12, e23-e41 (2018).
2. Lindahl, U., Couchman, J., Kimata, K. & Esko, J. D. Proteoglycans and Sulfated Glycosaminoglycans in Essentials of Glycobiology, 3rd edition, A. Varki, Ed. (Cold Spring Harbor Laboratory Press, Cold Spring Harbor, NY, 2017) Chapter 17.
3. Esko, J. D., Prestegard, J. H. & Linhardt, R. J. Proteins That Bind Sulfated Glycosaminoglycans Cold Spring Harbor (NY): Cold Spring Harbor Laboratory Press; 2015-2017. in Essentials of Glycobiology, 3rd edition, A. Varki, Ed. (Cold Spring Harbor Laboratory Press, Cold Spring Harbor, NY, 2017) Chapter 38.
4. DeAngelis, P. L., Liu, J. & Linhardt, R. J. Chemoenzymatic synthesis of glycosaminoglycans: Re-creating, re-modeling and re-designing nature's longest or most complex carbohydrate chains, Glycobiology 23, 764-777 (2013).
5. Zhang, X., Lin, L., Huang, H. & Linhardt, R. J. Chemoenzymatic synthesis of glycosaminoglycans, Acc. Chem. Res. 53, 335-346 (2020).
6. Liu, J. & Linhardt, R. J. Chemoenzymatic synthesis of heparan sulfate and heparin, Nat. Prod. Rep. 31, 1676-1685 (2020).
7. Otto, N. J., Solakyildirim, K., Linhardt, R. J., DeAngelis, P. L. Comamonas testosteronan synthase, a bifunctional glycosyltransferase that produces a unique heparosan polysaccharide analog. Glycobiology. 21(10):1331-1340 (2011).
8. DeAngelis, P. L., Otto, N. J., Methods of producing testosteronan polymers using testosteronan synthase. U.S. Pat. No. 9,695,427 (2017).
9. DeAngelis, P. L., Otto, N. J., Methods of producing testosteronan polymers using testosteronan synthase. U.S. Pat. No. 10,273,517 (2019).
10. Jia, L. & Ma, S. Recent advances in the discovery of heparanase inhibitors as anticancer agents, Eur. J. Med. Chem. 121, 209-220 (2016).
11. Heyman, B. & Yang, Y. Mechanisms of heparanase inhibitors in cancer therapy, Experimental Hematol. 44, 1002-1012 (2016).
12. Mayes, H. B., Broadbelt, L. J. & Beckham, G. T. How sugars pucker: Electronic structure calculations map the kinetic landscape of five biologically paramount monosaccharides and their implications for enzymatic catalysis. J. Am. Chem. Soc. 136, 1008-1022 (2014).
13. Wu, L. Viola, C. M., Brzozowski, A. M. & Davies, G. J. Structural characterization of human heparanase reveals insights into substrate recognition. Nat. Struct. & Molec. Biol. 22(12), 1016-1022 (2015).
14. Mohan C. D., Hari, S., Preetham, H. D., Rangappa, S., Barash, U., Ilan, N., Nayak, S. C. & Gupta, V. K., Basappa, Vlodavsky, I. & Rangappa, K. S. Targeting Heparanase in Cancer: Inhibition by Synthetic, Chemically Modified, and Natural Compounds. iScience 15, 360-390 (2019).
15. Sismey-Ragatz, A. E., Green, D. E., Otto, N. J., Rejzek, M., Field, R. A. & DeAngelis, P. L. Chemoenzymatic synthesis with distinct Pasteurella heparosan synthases: monodisperse polymers and unnatural structures. J. Biol. Chem. 282, 28321-28327 (2007).
16. Gilbert, E. E. The reactions of sulfur trioxide, and its adducts, with organic compounds. Chem. Rev. 62, 549-589 (1962)
17. Minamisawa, T., Asami, N., Fujita, H. & Suzuki, K. Method for sulfating glycosaminoglycans. U.S. Pat. No. 10,259,889 (2016)
18. Li, G., Masuko, S., Green, D. E., Xu, Y., Li, L., Zhang, F., Xue, C., Liu, J., DeAngelis, P. L., Linhardt, R. J. N-sulfotestosteronan, a novel substrate for heparan sulfate 6-O-sulfotransferases and its analysis by oxidative degradation. Biopolymers. 99(10):675-685 (2013)
19. Cowman, M. K., Chen, C. C., Pandya, M., Yuan, H., Ramkishun, D., LoBello, J., Bhilocha, S., Russell-Puleri, S., Skendaj, E. & Mijovic J. & Jing, W. Improved agarose gel electrophoresis method and molecular mass calculation for high molecular mass hyaluronan. Analytical Biochemistry 417, 50-56 (2011).
20. Otto N J, Green D E, Masuko S, Mayer A, Tanner M E, Linhardt R J, DeAngelis P L. J Biol Chem. 2012 Mar. 2; 287(10):7203-12. doi: 10.1074/jbc.M111.311704. Epub 2012 Jan. 10. PMID: 22235128.
21. Casu B., Torri G, Naggi A M, Giannini G, Pisano C, Penco S. Derivatives of partially desulphated glycosaminoglycans as heparanase inhibitors, endowed with antiangiogenic activity and devoid of anticoagulating effect. U.S. Pat. No. 7,781,416 (2010).

1. A polymer, comprising the repeat structure [4-D-glucuronic acid-α1,4-D-N-acetylglucosamine-α1-]ₙ ([-4-D-GlcUA-α1,4-D-GlcNAc-α1-]ₙ), wherein at least one sulfur moiety is linked to at least one hydroxyl of the repeat structure, and wherein n is in a range of from about 2 to about 500.

2. The polymer of claim 1, wherein the sulfur moiety is SO₃.

3. The polymer of claim 1, comprising the structure O-Sulfo [-4-GlcUA-α1,4-GlcNAc-α1-]ₙ.

4. The polymer of claim 1, wherein the sulfur moiety is O-linked to the GlcNAc.

5. The polymer of claim 1, wherein the sulfur moiety is O-linked to the GlcUA.

6. A composition, comprising the polymer of claim 1.

7. The composition of claim 6, wherein the composition is in an aqueous solution.

8. A composition, comprising:
at least one polymer of claim 1; and
a heparosan chain or other polymer linked thereto.

9. A method of inhibiting heparanase activity, comprising exposing the heparanase to at least one polymer of claim 1.

10. A method of inhibiting heparanase activity, comprising exposing the heparanase to at least one composition of claim 6.

11. The method of claim 9, wherein the heparanase activity is inhibited in vitro.

12. The method of claim 9, wherein the heparanase activity is inhibited in vivo.

13. A pharmaceutical composition, comprising:
at least one polymer of claim 1; and
a pharmaceutically acceptable excipient.

14. A method of treating a subject in need of treatment, comprising the step of:
administering the pharmaceutical composition of claim 13 to the subject in need of treatment.

15. The method of claim 14, wherein the subject has cancer or is predisposed to cancer.

16. The method of claim 14, wherein the subject has at least one disease or condition associated with overexpression, misregulation, or hyperactivity of heparanase.

17. The method of claim 16, wherein the at least one disease or condition is selected from the group consisting of diabetes, a complication of diabetes, atherosclerosis, thrombosis, a viral infection, and combinations thereof.

18. A method of producing a sulfated testan polymer, the method comprising the steps of:
producing a polymer backbone having the repeat structure [4-D-glucuronic acid-α1,4-D-N-acetylglucosamine-α1-]ₙ ([-4-D-GlcUA-α1,4-D-GlcNAc-α1-]ₙ), wherein n is in a range of from about 2 to about 500; and
linking at least one sulfur moiety to at least one hydroxyl of the repeat structure.

19. The method of claim 18, wherein the polymer backbone is produced by native fermentation or recombinant production using an organism expressing a testosteronan synthase gene.

20. The method of claim 18, wherein the polymer backbone is produced by chemical or chemoenzymatic synthesis.

21. A kit, comprising at least one polymer of claim 1.

22-23. (canceled)
